(19) United States
(12) Patent Application Publication
(10) Pub. No.: US 2025/0228643 A1
(43) Pub. Date: Jul. 17, 2025

(54) LEFT ATRIAL APPENDAGE CLOSURE DEVICE IMPLANTATION WITH VISUALIZATION

(71) Applicant: Boston Scientific Scimed, Inc., Maple Grove, MN (US)

(72) Inventors: Carly Rogers, Eden Prairie, MN (US); David John Onushko, Maple Grove, MN (US)

(73) Assignee: Boston Scientific Scimed, Inc., Maple Grove, MN (US)

(21) Appl. No.: 19/017,178

(22) Filed: Jan. 10, 2025

Related U.S. Application Data
(60) Provisional application No. 63/620,388, filed on Jan. 12, 2024.

(51) Int. Cl. A61B 90/00; A61B 17/00

(52) U.S. Cl. CPC A61B 90/36; A61B 17/00234; A61B 2017/00106; A61B 2017/00292; A61B 2017/00876; A61B 2090/3782

(57) ABSTRACT

A first elongate member is adapted to be advanced through a puncture formed within a body structure such as a septum and a second elongate member is adapted to be advanced through the puncture adjacent the first elongate member. The first elongate member includes a first elongate shaft and a ferrous material disposed within the first elongate shaft at a position intended to remain at a first side of the puncture site. The second elongate member includes a second elongate shaft and one or more magnets secured to the second elongate shaft that are adapted to be attracted to the ferrous material of the first elongate shaft in order to guide the second elongate member towards the first elongate member and through the puncture site. The first elongate member may be a guidewire and the second elongate member may be a visualization catheter.

### CROSS REFERENCE TO RELATED APPLICATIONS

This application claims the benefit of priority of U.S. Provisional Application No. 63/620,388 filed Jan. 12, 2024, the entire disclosure of which is hereby incorporated by reference.

### TECHNICAL FIELD

The disclosure pertains to occlusive medical devices for occluding the left atrial appendage and more particularly to delivering occlusive medical devices to the left atrial appendage with visualization.

### BACKGROUND

A wide variety of medical devices have been developed for medical use including, for example, devices for occluding the left atrial appendage. Devices for occluding the left atrial appendage may be delivered through the atrial septum, and implantation may include visualization techniques such as using an ICE (Intracardiac Echocardiography) catheter, which may include advancing the ICE catheter through the atrial septum. Of the known medical devices and methods, each has certain advantages and disadvantages. There is an ongoing need to provide alternative medical devices as well as alternative methods for manufacturing and using the medical devices.

### SUMMARY

This disclosure provides design, material, manufacturing method, and use alternatives for medical devices. An example may be found in a medical device assembly. The medical device assembly includes a first elongate member adapted to be advanced through a puncture site formed within a body structure and a second elongate member adapted to be advanced through the puncture site adjacent the first elongate member. The first elongate member includes a first elongate shaft and a ferrous material within the first elongate shaft at a position intended to remain at a first side of the puncture site. The second elongate member includes a second elongate shaft and one or more magnets secured to the second elongate shaft, the one or more magnets adapted to be attracted to the ferrous material of the first elongate member in order to guide the second elongate member towards the first elongate member and through the puncture site.

Alternatively or additionally, the ferrous material may include an additional magnet.

Alternatively or additionally, the body structure may include a cardiac septum, and the puncture site may be formed in the cardiac septum.

Alternatively or additionally, the body structure may include an atrial septum, and the puncture site may be formed in the atrial septum.

Alternatively or additionally, the additional magnet may include an elongate magnet extending along the first elongate shaft.

Alternatively or additionally, the first elongate shaft may include a coiled elongate shaft, and the additional magnet may be coiled within the coiled elongate shaft.

Alternatively or additionally, the additional magnet may include a single magnet secured to the first elongate shaft.

Alternatively or additionally, the additional magnet may include a plurality of magnets secured to the first elongate shaft.

Alternatively or additionally, the one or more magnets may include one or more electromagnets.

Alternatively or additionally, the first elongate member may include a guidewire.

Alternatively or additionally, the second elongate member may include a visualization catheter.

Alternatively or additionally, the second elongate member may include an intracardiac echocardiographic (ICE) catheter.

Alternatively or additionally, the second elongate member may include a guidewire.

Another example may be found in an assembly for accessing and visualizing a patient's left atrium. The assembly includes a guidewire and a visualization catheter. The guidewire includes a guidewire shaft adapted to extend through a puncture formed in the patient's atrial septum and a guidewire magnet secured to the guidewire shaft at a position intended to remain at a first side of the puncture. The visualization catheter includes an elongate shaft and one or more visualization catheter magnets secured to the elongate shaft, the one or more visualization catheter magnets adapted to be attracted to the guidewire magnet in order to guide the visualization catheter towards the guidewire and through the puncture.

Alternatively or additionally, the guidewire magnet may include an elongate magnet extending along the guidewire shaft.

Alternatively or additionally, the guidewire magnet may include a single magnet secured to the guidewire shaft.

Alternatively or additionally, the guidewire magnet may include a plurality of magnets secured relative to the guidewire shaft.

Alternatively or additionally, the visualization catheter may include an intracardiac echocardiographic (ICE) catheter.

Another example may be found in a kit for accessing and visualizing a patient's left atrium. The kit includes a guidewire having a guidewire magnet secured to the guidewire at a position intended to remain at a first side of a puncture formed in the atrial septum and a visualization catheter adapted to be advanced through the puncture adjacent the guidewire, the visualization catheter including an elongate shaft and one or more visualization catheter magnets secured to the elongate shaft, the one or more visualization catheter magnets adapted to be attracted to the guidewire magnet in order to guide the visualization catheter towards the guidewire and through the puncture.

Alternatively or additionally, the one or more visualization catheter magnets may include electromagnets.

Alternatively or additionally, the visualization catheter may include an intracardiac echocardiographic (ICE) catheter.

Another example may be found in a medical device assembly that includes a first guidewire and a second guidewire. The first guidewire may include a funnel that is secured to the first guidewire at a position that will place the funnel proximate an atrial septum puncture site when the first guidewire is advanced into and through the atrial septum puncture site. The second guidewire, when advanced, may be adapted to be captured by the funnel and thus guided towards the atrial septum puncture site. The funnel may be tapered from a large proximal opening adapted to capture the second guidewire to a small distal opening adapted to fit into the atrial septum puncture site and thus guide the second guidewire into and through the atrial septum puncture site.

Alternatively or additionally, the funnel may be expandable from a collapsed configuration to an expanded configuration.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a schematic view of a human heart;

FIGS. 2A through 2K are schematic views showing a procedure for penetrating the atrial septum under visualization and to access the left atrial appendage in order to implant a left atrial appendage closure device within the left atrial appendage;

FIG. 3 is a schematic view of an illustrative guidewire usable in the procedure shown in FIGS. 2A through 2K;

FIG. 4 is a schematic view of an illustrative guidewire usable in the procedure shown in FIGS. 2A through 2K;

FIG. 5 is a schematic view of an illustrative guidewire usable in the procedure shown in FIGS. 2A through 2K;

FIG. 6A is a schematic view of an illustrative guidewire usable in the procedure shown in FIGS. 2A through 2K;

FIG. 6B is a cross-sectional view taken along the line 6B-6B of FIG. 6A;

FIG. 7 is a schematic view of an illustrative visualization catheter usable in the procedure shown in FIGS. 2A through 2K;

FIG. 8 is a schematic view of an illustrative visualization catheter usable in the procedure shown in FIGS. 2A through 2K;

FIG. 9 is a schematic view of an illustrative visualization catheter usable in the procedure shown in FIGS. 2A through 2K;

FIG. 10 is a schematic view showing an illustrative method for accessing the left atrium;

FIG. 11 is a schematic view showing an illustrative method for accessing the left atrium; and

FIGS. 12 and 13 are schematic views of an illustrative delivery device for delivering a left atrial appendage closure device.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

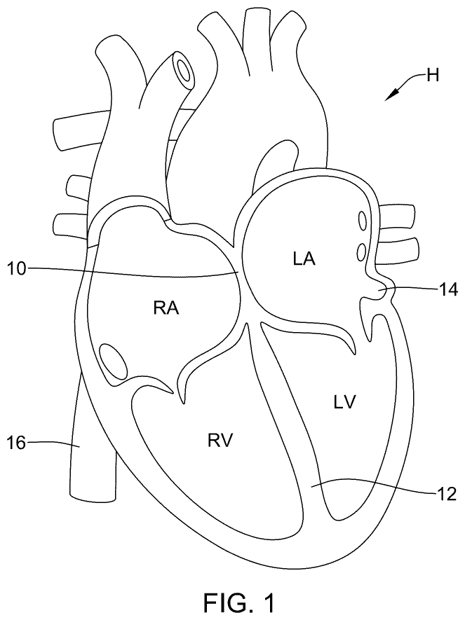

FIG. 1 is a schematic view of a human heart H. The primary chambers of the heart H include a right atrium RA, a right ventricle RV, a left atrium LA and a left ventricle LV. The right atrium RA and the left atrium LA are separated by an atrial septum 10 while the right ventricle RV and the left ventricle LV are separated by a ventricular septum 12. In some instances, the atrium septum 10 and the ventricular septum 12 are together generally referred to as an atrioventricular septum. The left atrium LA includes a left atrial appendage (LAA) 14. An inferior vena cava 16 is fluidly coupled with the right atrium RA.

The LAA 14 is a small sac attached to the left atrium LA of the heart H as a pouch-like extension. In some patients, the LAA 14 may not properly contract with the left atrium LA, causing stagnant blood to pool within its interior, which can lead to the undesirable formation of thrombi within the LAA 14. Thrombi forming in the LAA 14 may break loose from this area and enter the blood stream. Thrombi that migrate through the blood vessels may eventually plug a smaller vessel downstream and thereby contribute to stroke. As a treatment, medical devices have been developed which are positioned in the LAA 14 and deployed to close off the ostium of the LAA 14. Over time, the exposed surface(s) spanning the ostium of the LAA 14 becomes covered with tissue (a process called endothelization), effectively removing the LAA 14 from the circulatory system and reducing or eliminating the number of thrombi which may enter the blood stream from the LAA 14.

In some instances, the left side of the heart H may be reached by accessing the right side of the heart H and then penetrating the atrial septum 10 and/or the ventricular septum 12 in order to access the left side of the heart H. As an example, the left atrium LA may be reached by first accessing the right atrium RA and then penetrating through the atrial septum 10. Once the left atrium LA has been reached, it is possible to reach the LAA 14 and to implant an occlusive device within the LAA 14. A puncture may be formed within the atrial septum 10. In some instances, a first elongate medical device such as a guidewire may be extended through the puncture. There may be a desire to advance a second elongate medical device such as a visualization catheter through the same puncture within the atrial septum 10 in order to avoid unnecessary trauma to the atrial septum 10. However, getting the second elongate medical device positioned to be able to pass through the same puncture within the atrial septum 10 as the first elongate medical device can be problematic.

In some instances, the first elongate medical device and the second elongate medical device may each include structure that facilitates the second elongate medical device to find or otherwise be attracted to the first elongate medical device. In some instances, the first elongate medical device may include a ferrous material and the second elongate medical device may include one or more magnets that are attracted to the ferrous material. In some cases, the ferrous material may include one or more additional magnets. In some instances, the additional magnets forming part of the first elongate medical device may be natural magnets such as rare earth magnets. In some instances, the magnet or magnets forming part of the second elongate medical device may be natural magnets such as rare earth metals. The magnet or magnets forming part of the second elongate medical device may also be electromagnets, for example. Once the first elongate medical device has been advanced through the puncture formed within the atrial septum 10, the magnet or magnets included with the second elongate medical device will be attracted to the ferrous material of the first elongate medical device, and/or to the additional magnet or magnets included with the first elongate medical device once the second elongate medical device is positioned close enough to the first elongate medical device for attractive magnetic forces to influence the position of the second elongate medical device relative to the first elongate medical device.

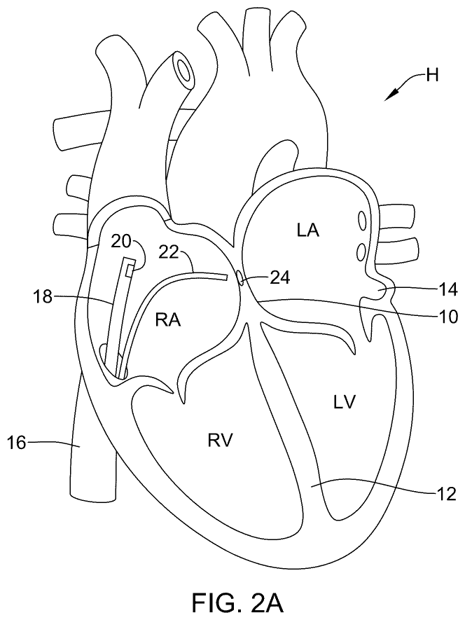

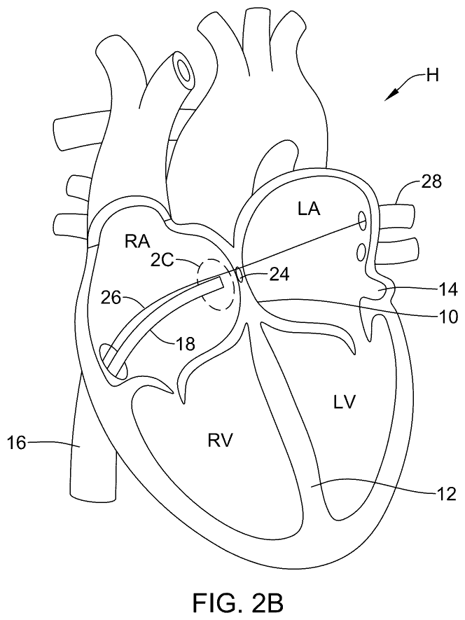

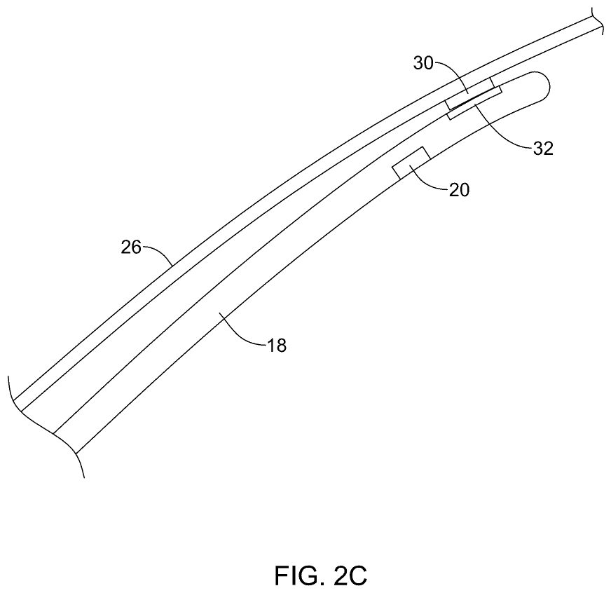

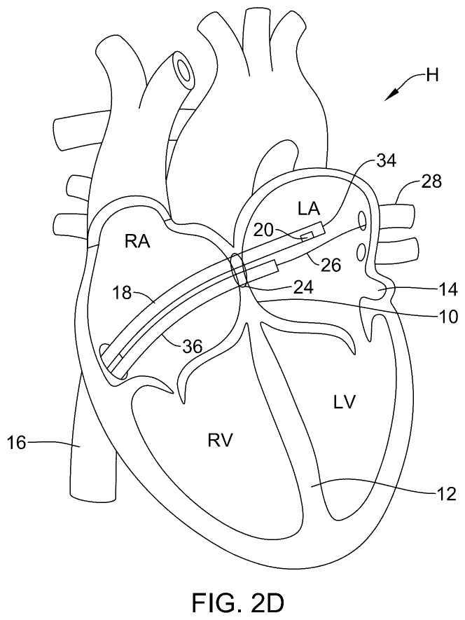

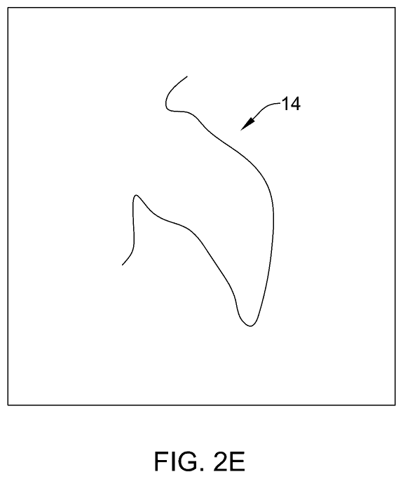

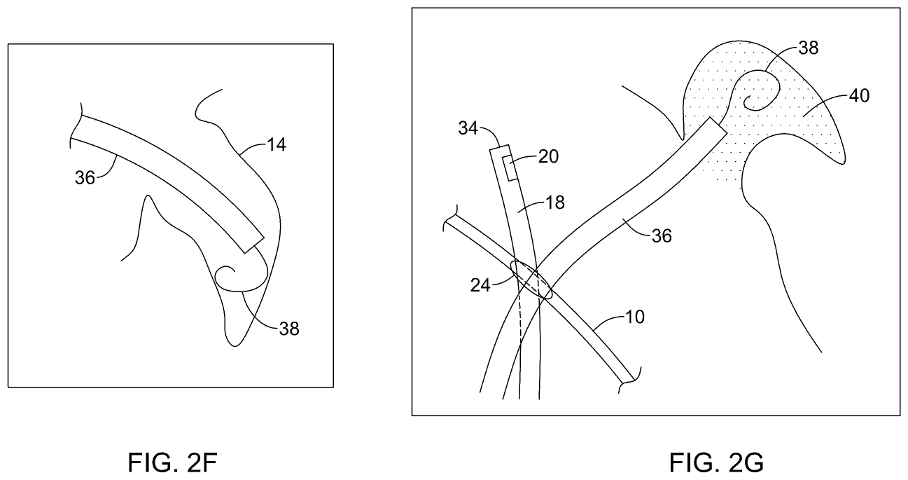

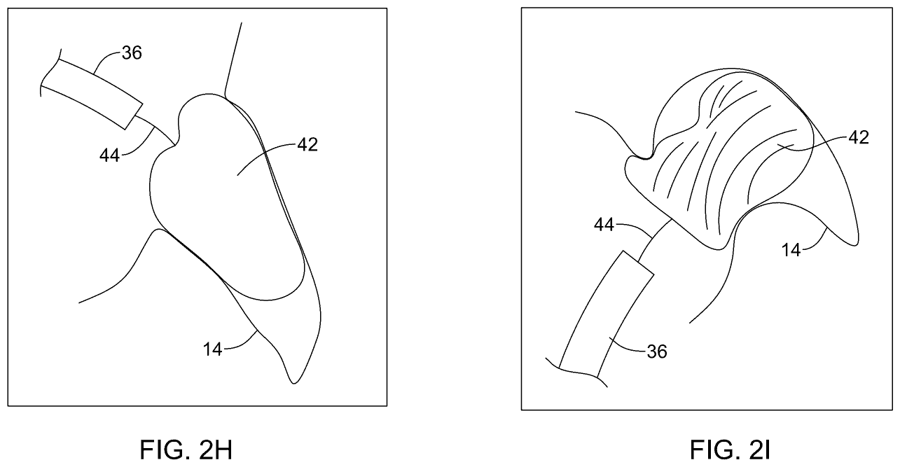

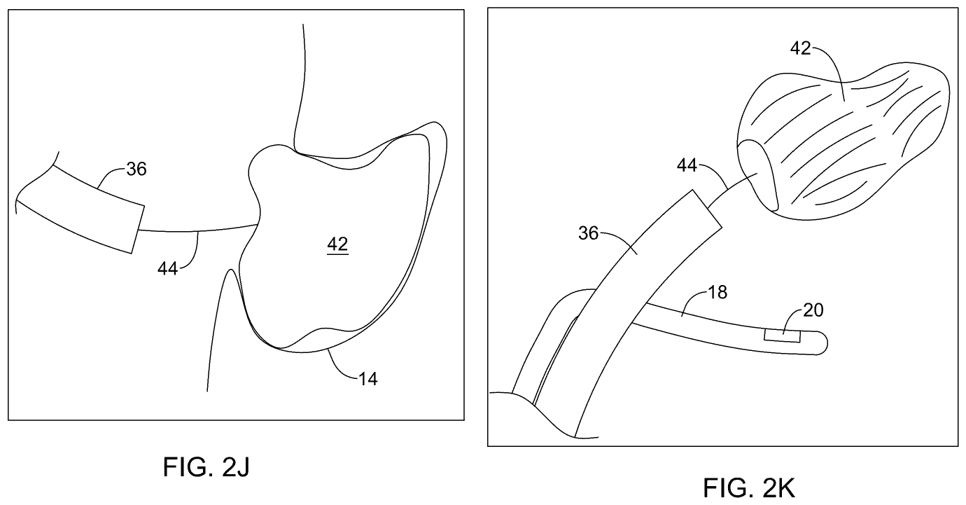

FIGS. 2A through 2K provide schematic views of using a guidewire as the first elongate medical device to assist in locating a visualization catheter as the second elongate medical device such that the visualization catheter is able to proceed through the same puncture within the atrial septum 10 that the guidewire extends through. As will be discussed with respect to subsequent Figures, the guidewire includes a guidewire magnet and the visualization catheter includes one or more visualization catheter magnets that are attracted to the guidewire magnet of the guidewire.

In FIG. 2A, a visualization catheter 18 has been advanced through the inferior vena cava 16 and into the right atrium RA. In some instances, the visualization catheter 18 may be an intracardiac echocardiographic (ICE) catheter, although other types of visualization catheters are also contemplated. When the visualization catheter 18 is an ICE catheter, the visualization catheter 18 may include a camera 20 (shown schematically). In some instances, the visualization catheter 18 may be used to visualize using a needle 22 to form an puncture 24 that extends through the atrial septum 10. Being able to visualize the atrial septum 10 using the visualization catheter 18 enables the person using the needle 22 to form the puncture 24 to select an optimal location within the atrial septum 10 to form the puncture 24. The relative location of the puncture 24 may be important when guiding additional components through the puncture 24 and into the left atrium LA.

Once the puncture 24 has been formed within the atrial septum 10 under visual guidance, a guidewire 26 may be advanced through the puncture 24, as seen in FIG. 2B. In some instances, the guidewire 26 may be advanced through the puncture 24 and into the left atrium LA. In some instances, the guidewire 26 may be advanced such that the guidewire 26 extends into the left upper pulmonary vein (LUPV) 28. The visualization catheter 18 may be manipulated to guide the visualization catheter 18 closer to the guidewire 26. FIG. 2C is an enlarged view, showing that the guidewire 26 includes a guidewire magnet 30 and that the visualization catheter 18 includes a visualization catheter magnet 32. The guidewire magnet 30 may be a natural magnet such as a rare earth magnet. The visualization catheter magnet 32 may be a natural magnet such as a rare earth magnet. The visualization catheter magnet 32 may be an electromagnet. The camera 20 may be disposed on an opposing side from the visualization catheter magnet 32, and thus is not visible in the illustrated orientation.

The guidewire magnet 30 may be oriented to have an outward-facing portion having a first polarity and the visualization catheter magnet 32 may be oriented to have an outward-facing portion having a second polarity that is opposite that of the first polarity such that the guidewire magnet 30 and the visualization catheter magnet 32 will exhibit an attractive force between the guidewire magnet 30 and the visualization catheter magnet 32 that helps the visualization catheter 18 move towards the guidewire 26 so that the visualization catheter 18 may be advanced along the guidewire 26 and through the puncture 24. While the guidewire magnet 30 is schematically shown as a single magnet, and the visualization catheter magnet 32 is schematically shown as a single magnet, this is just an example. The guidewire magnet 30 may be a single magnet or a number of magnets that are spaced apart along the guidewire 26, for example. The visualization catheter magnet 32 may be a single magnet or two or more magnets that are spaced apart from each other. In some instances, the visualization catheter magnet 32 may be an electromagnet that can be selectively actuated by applying electrical power to the visualization catheter magnet 32 when it is desired for the visualization catheter magnet 32 to function as a magnet. When no power is supplied to the electromagnet, the visualization catheter magnet 32 does not function as a magnet and thus may provide reduced interference with the camera 20.

As seen in FIG. 2D, the visualization catheter 18 has been advanced into the left atrium LA such that a distal end 34 of the visualization catheter 18 is close to the LUPV 28. A therapeutic catheter 36 has been advanced over the guidewire 26, through the puncture 24 and into the left atrium LA. Alternatively, the guidewire 26 may be withdrawn proximally before the therapeutic catheter 36 is advanced into the left atrium LA. In some instances, the therapeutic catheter 36 may be adapted to deploy a left atrial appendage closure (LAAC) device within the LAA 14. As noted, the distal end 34 of the visualization catheter 36 is positioned within the LAA 14 and is close to the LUPV 28. As a result, the visualization catheter 36 is positioned to be able to visualize the LAA 14. FIG. 2E provides a schematically-shown example view of the LAA 14 that may be captured when the visualization catheter 26 is an ICE catheter. FIG. 2F provides an schematically-shown example view of the LAA 14, captured by an ICE catheter, of the therapeutic catheter 36 advanced into the LAA 14. A pigtail catheter 38 may be seen as extending distally from the therapeutic catheter 36.

In FIG. 2G, the visualization catheter 18 is positioned to visualize the LAA 14. The therapeutic catheter 36 is still positioned within the LAA 14, and the pigtail catheter 38 has released a contrast fluid 40, illustrated as a dotted pattern, within the LAA 14. The image shown in FIG. 2G may be considered as being obtained under fluoroscopic imaging. As noted, in some instances the therapeutic catheter 36 may be adapted to deploy a LAAC device within the LAA 14.

FIG. 2H shows a corresponding schematically-shown image as captured by an ICE catheter as the visualization catheter 18. An LAAC device 42 that is attached to a wire 44 extending from the therapeutic catheter 36 is shown as positioned within the LAA 14 and is in fact expanded to largely fill the LAA 14. FIG. 2I shows a corresponding schematically-shown fluoroscopic image of the LAA 14 and the LAAC device 42 disposed within the LAA 14. FIG. 2J provides another schematically-shown ICE image shown in a supramitral view and FIG. 2K provides another schematically-shown fluoroscopic image shown in a supramitral view. In FIG. 2K, the therapeutic catheter 36 is shown positioned to capture the image shown in FIG. 2J. After implantation, color Doppler may be performed to ascertain sealing performance between the LAAC device 42 and the LAA 14. In some instances, shoulder to shoulder measurements may be performed in order to ascertain the degree of device compression. Sealing performance and device compression determinations may be performed before finally releasing the LAAC device 42 from the therapeutic agent

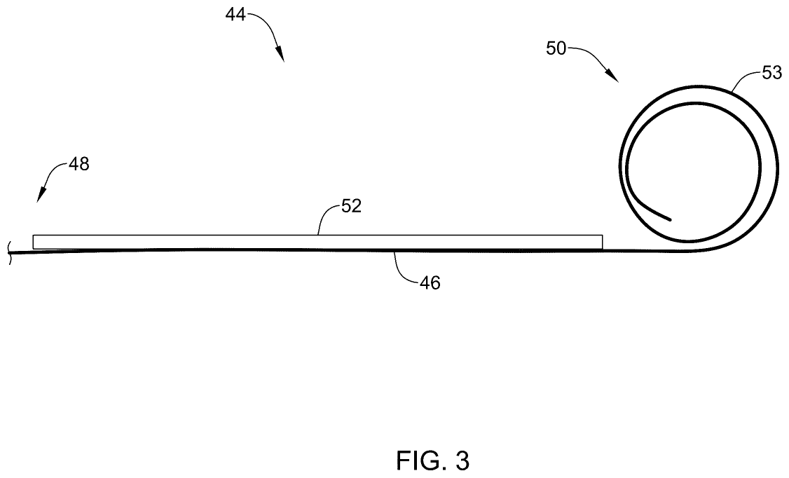

As noted, the first elongate medical device (such as the guidewire 26) may include one or more guidewire magnets 30. FIG. 3 is a schematic view of an illustrative guidewire 44 that may be considered as being an example of the guidewire 26. The illustrative guidewire 44 includes an elongate shaft 46 extending from a proximal region 48 to a distal region 50. An elongate magnet 52 is secured to the elongate shaft 46. In some instances, the elongate magnet 52 may be secured to the elongate shaft 46 near the distal region 50 at a position that would correspond to the elongate magnet 52 being positioned on a first side of the atrial septum 10, such as near the atrial septum 10 but remaining within the right atrium RA. In some instances, the distal region 50 of the elongate shaft 46 may include an atraumatic coiled tip 52, but this is not required in all cases. The elongate magnet 52 may have a length sufficient to allow the one or more second magnets 32 (of the visualization catheter 18) to remain proximate the elongate magnet 52 while the visualization catheter 18 is slid distally along the guidewire 44 while advancing the visualization catheter 18 towards and through the puncture 24 (shown in FIG. 2A).

The elongate magnet 52 may have a length in a range of 0.1 inches to 24 inches and a diameter of up to 0.5 inches. In some instances, the elongate magnet 52 may have a length of 1 millimeter to 50 millimeters and a diameter ranging from 0.05 inches to 0.050 inches. The elongate magnet 52 may be secured to the elongate shaft 46 in any suitable fashion, including adhesively securing the elongate magnet 52 in place. In some instances, the elongate magnet 52 may be welded or soldered in position. In some instances, a thin polymeric layer (not shown), such as a shrink-wrap layer, may be used to hold the elongate magnet 52 in position relative to the elongate shaft 46. As shown, the elongate magnet 52 is aligned axially with, but next to, the elongate shaft 46. In some instances, the elongate magnet 52 may have a cylindrical shape, and the elongate shaft 46 may extend coaxially within the elongate magnet 52. While shown as a single element, the elongate magnet 52 may include a number of individual magnets that are placed end to end in position relative to the elongate shaft 46. In some instances, the elongate shaft 46 may include a ferrous material such as a steel that the second magnet or magnets in the visualization catheter 18 will be attracted to. In such cases, the elongate magnet 52 may be omitted.

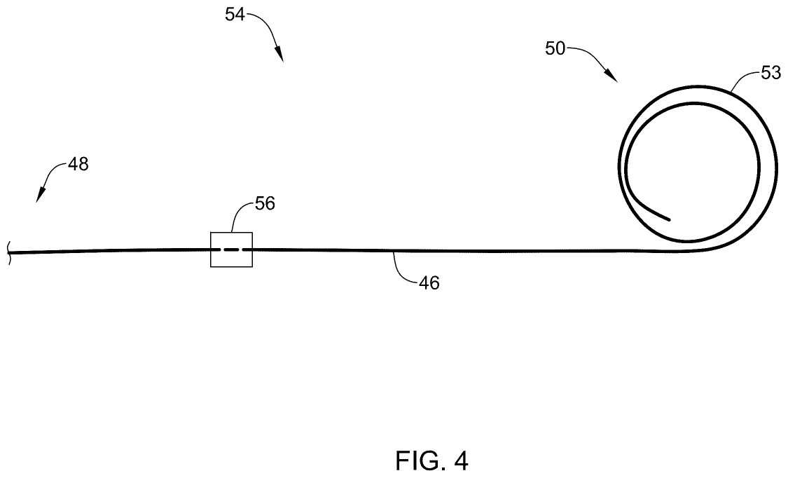

FIG. 4 is a schematic view of an illustrative guidewire 54 that may be considered as being an example of the guidewire 26. The illustrative guidewire 54 includes an elongate shaft 46 extending from a proximal region 48 to a distal region 50. A guidewire magnet 56 is secured to the elongate shaft 46. In some instances, the guidewire magnet 56 may be secured to the elongate shaft 46 near the distal region 50 at a position that would correspond to the guidewire magnet 56 being positioned on a first side of the atrial septum 10, such as near the atrial septum 10 but remaining within the right atrium RA. In some instances, the distal region 50 of the elongate shaft 46 may include an atraumatic coiled tip 52, but this is not required in all cases. The guidewire magnet 56 may have a length sufficient to allow the one or more visualization catheter magnets 32 to be attracted to the guidewire magnet 56 as the visualization catheter 18 is moved towards the guidewire 54.

The guidewire magnet 56 may be secured to the elongate shaft 46 in any suitable fashion, including adhesively securing the guidewire magnet 56 in place. In some instances, the guidewire magnet 56 may be welded or soldered in position. In some instances, a thin polymeric layer (not shown), such as a shrink-wrap layer, may be used to hold the guidewire magnet 56 in position relative to the elongate shaft 46. As shown, the guidewire magnet 56 may have a cylindrical shape, and the elongate shaft 46 may extend coaxially within the guidewire magnet 56. In some instances, the guidewire magnet 56 may instead be aligned axially with, but next to, the elongate shaft 46.

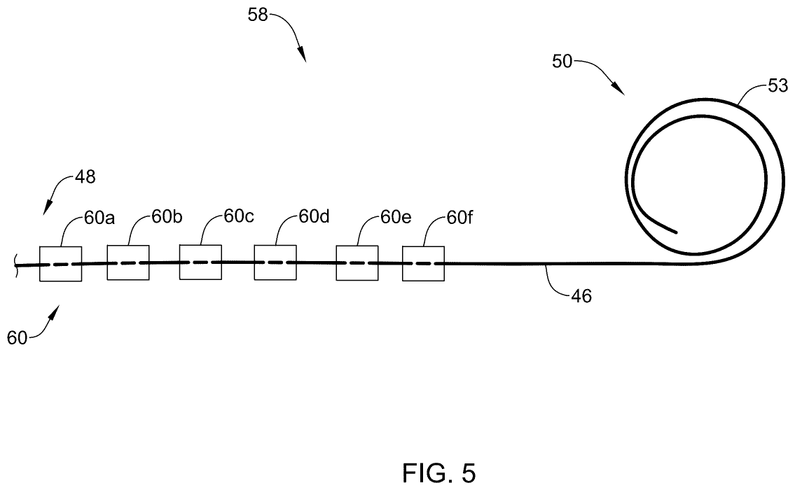

FIG. 5 is a schematic view of an illustrative guidewire 58 that may be considered as being an example of the guidewire 26. The illustrative guidewire 58 includes an elongate shaft 46 extending from a proximal region 48 to a distal region 50. A plurality of guidewire magnets 60, individually labeled as 60a, 60b, 60c, 60d, 60e and 60f, are secured to the elongate shaft 46. In some instances, the guidewire magnets 60 may be secured to the elongate shaft 46 near the distal region 50 at a position that would correspond to at least some of the guidewire magnets 60 being positioned on a first side of the atrial septum 10, such as near the atrial septum 10 but remaining within the right atrium RA. In some instances, the distal region 50 of the elongate shaft 46 may include an atraumatic coiled tip 52, but this is not required in all cases. The guidewire magnets 60 may be arranged to allow the one or more visualization catheter magnets 32 to be attracted to successive magnets of the guidewire magnets 60 as the visualization catheter 18 is moved towards the guidewire 54.

The guidewire magnets 60 may be secured to the elongate shaft 46 in any suitable fashion, including adhesively securing the guidewire magnets 60 in place. In some instances, the guidewire magnets 60 may be welded or soldered in position. In some instances, a thin polymeric layer (not shown), such as a shrink-wrap layer, may be used to hold the guidewire magnets 60 in position relative to the elongate shaft 46. As shown, the guidewire magnets 60 may have a cylindrical shape, and the elongate shaft 46 may extend coaxially within the guidewire magnets 60. In some instances, the guidewire magnets 60 may instead be aligned axially with, but next to, the elongate shaft 46.

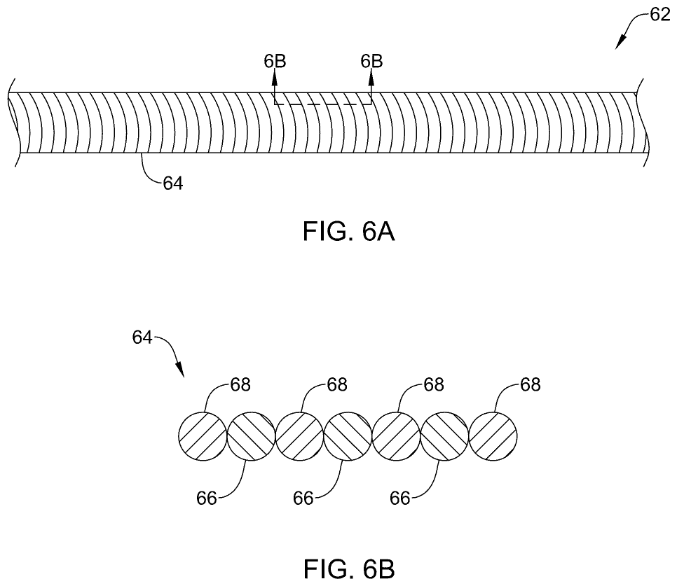

FIG. 6A is a schematic view of a portion of an illustrative guidewire 62 that may be considered as being an example of the guidewire 26 and FIG. 6B is a cross-sectional view taken along the line 6B-6B of Figure A, showing the construction of an elongate shaft 64 forming the guidewire 62. As can be seen, the elongate shaft 64 is a coiled construction. In some instances, one or more guidewire magnets 30 may be secured relative to the elongate shaft 64. In some instances, the one or more guidewire magnets 30 may be coiled within the coiled construction. As can be seen in FIG. 6B, a coiled magnet 66 may be seen as being co-coiled together with a metal coil 68. The metal coil 68 may be formed of stainless steel, for example. In some instances, the metal coil 68 may be formed of a shape memory metal such as Nitinol. In some instances, the coiled magnet 66 may extend a substantial length of the elongate shaft 64. In some instances, the coiled magnet 66 may only extend along a portion of the elongate shaft 64 that is intended to be positioned on a first side of the atrial septum 10, such as near the atrial septum 10 but remaining within the right atrium RA. The coiled magnet 66 may have a length sufficient to allow the one or more second magnets 32 (of the visualization catheter 18) to be attracted to the coiled magnet 66 as the visualization catheter 18 is moved towards the guidewire 54.

In some cases, the coiled magnet 66 may be a rare earth magnet. In some cases, the coiled magnet 66 may be an electromagnet that becomes magnetic when an electrical current passes through the coiled magnet 66. In some cases, the elongate shaft 64 may include a magnetic core that disposed within the elongate shaft 64, in place of the coiled magnet 66. As an example, the elongate shaft 64 may include a cylindrical magnet that is disposed within the elongate shaft 64. The elongate shaft 64 may include a ferrous insert, instead of a cylindrical magnet. These are just examples.

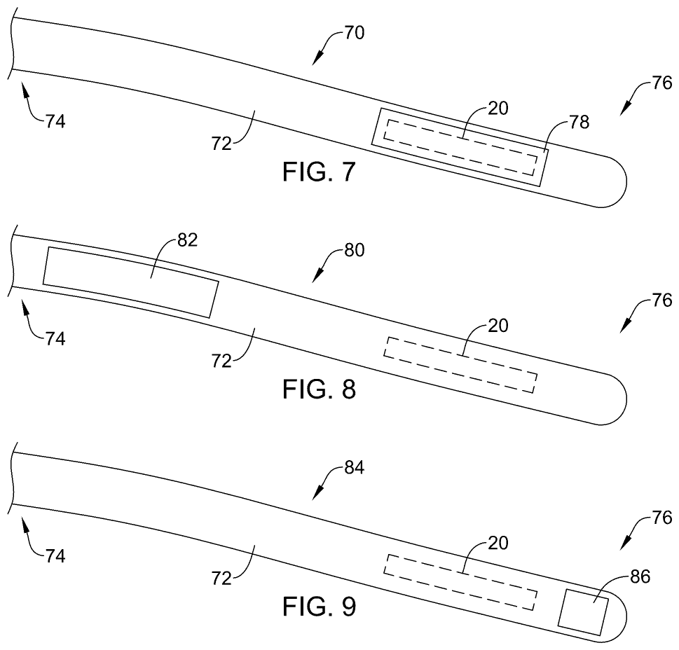

FIG. 7 is a schematic view of an illustrative ICE catheter 70 that may be considered as being an example of the visualization catheter 18. The illustrative ICE catheter 70 includes an elongate shaft 72 that houses the camera 20 (shown in phantom as the camera 20 is on the opposite side in the illustrated orientation) as well as the electrical connectors necessary for the camera 20. The ICE catheter 70 includes an ICE catheter magnet 78 that is secured to the elongate shaft 72 within the distal region 76 of the elongate shaft 72. As shown, the ICE catheter magnet 78 is positioned on a side of the elongate shaft 72 that is opposite that of the camera 20 so as to not interfere with the camera 20. While a single ICE catheter magnet 78 is shown, in some instances the ICE catheter 70 may include two or more ICE catheter magnets 78. In some instances, the ICE catheter magnet 78 is a natural magnet such as a rare earth magnet. In some instances, the ICE catheter magnet 78 may be an electromagnet that can be selectively actuated when needed by providing electrical power to the electromagnet.

FIG. 8 is a schematic view of an illustrative ICE catheter 80 that may be considered as being an example of the visualization catheter 18. The illustrative ICE catheter 80 includes an elongate shaft 72 that houses the camera 20 (shown in phantom as the camera 20 is on the opposite side in the illustrated orientation) as well as the electrical connectors necessary for the camera 20. The ICE catheter 80 includes an ICE catheter magnet 82 that is secured to the elongate shaft 72 within the distal region 76 of the elongate shaft 72, but more proximally relative to the position of the ICE catheter magnet 78 shown in FIG. 7. As shown, the ICE catheter magnet 82 is positioned on a side of the elongate shaft 72 that is opposite that of the camera 20 so as to not interfere with the camera 20. While a single ICE catheter magnet 82 is shown, in some instances the ICE catheter 80 may include two or more ICE catheter magnets 82. In some instances, the ICE catheter magnet 82 is a natural magnet such as a rare earth magnet. In some instances, the ICE catheter magnet 82 may be an electromagnet that can be selectively actuated when needed by providing electrical power to the electromagnet.

FIG. 9 is a schematic view of an illustrative ICE catheter 84 that may be considered as being an example of the visualization catheter 18. The illustrative ICE catheter 84 includes an elongate shaft 72 that houses the camera 20 (shown in phantom as the camera 20 is on the opposite side in the illustrated orientation) as well as the electrical connectors necessary for the camera 20. The ICE catheter 84 includes an ICE catheter magnet 86 that is secured to the elongate shaft 72 within the distal region 76 of the elongate shaft 72 near the distal end thereof. As shown, the ICE catheter magnet 86 is positioned on a side of the elongate shaft 72 that is opposite that of the camera 20 so as to not interfere with the camera 20. In some instances, the ICE catheter magnet 86 is a natural magnet such as a rare earth magnet. In some instances, the ICE catheter magnet 86 may be an electromagnet that can be selectively actuated when needed by providing electrical power to the electromagnet. In some instances, the ICE catheter magnet 86 may include a proximal electromagnet and a smaller distal electromagnet to aid in targeting the septum crossing point.

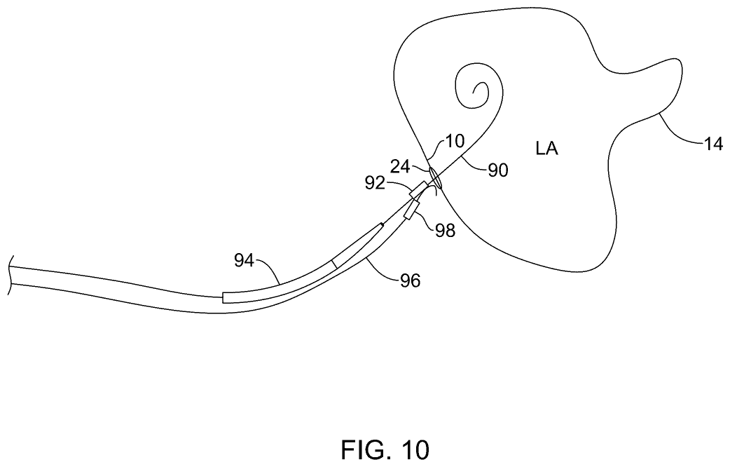

FIG. 10 is a schematic view showing an example of wire to wire guidance. A first guidewire 90 has been advanced through the puncture 24 formed within the atrial septum 10. The first guidewire 90 includes a first guidewire magnet 92. In some instances, a sheath 94 may be advanced over the first guidewire 90, but this is not required in all cases. A second guidewire 96 including a second guidewire magnet 98 may be guided towards the first guidewire 90 such that the second guidewire magnet 98 is attracted to the first guidewire magnet 92. Once the second guidewire 96 is advanced through the puncture 24 and into the left atrium LA, the second guidewire 96 may be used as a rail for advancing any desired medical device such as a secondary sheath or a mono-rail style visualization catheter. In some instances, an ICE catheter may be adapted to be adapted for advancement over the second guidewire 96.

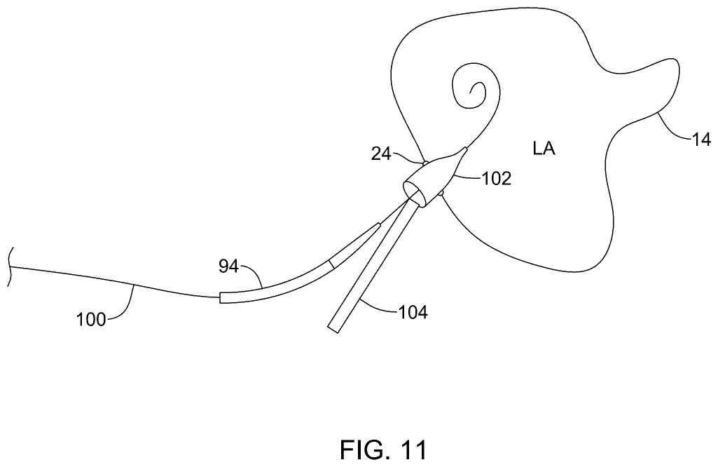

FIG. 11 is a schematic view showing an example of a bulls-eye lead-in for crossing the atrial septum 10. A first guidewire 100 has been advanced through the puncture 24 formed within the atrial septum 10. The sheath 94 may be advanceable over the first guidewire 100, for example. The first guidewire 100 includes a funnel 102 that is secured to the first guidewire 100. In some instances, the funnel 102 may be adapted to be positioned within the puncture 24. A medical device 104 may be advanced towards, into and through the funnel 102 in order to guide the medical device 104 through the puncture 24 and into the left atrium LA. The funnel 102 may be a sleeve or a basket that may be expanded from a collapsed configuration to an expanded configuration, for example. The funnel 102 may include a braided, polymeric or Nitinol element such as a lasso to help keep the funnel 102 open. In some instances, the element may be an inflatable balloon.

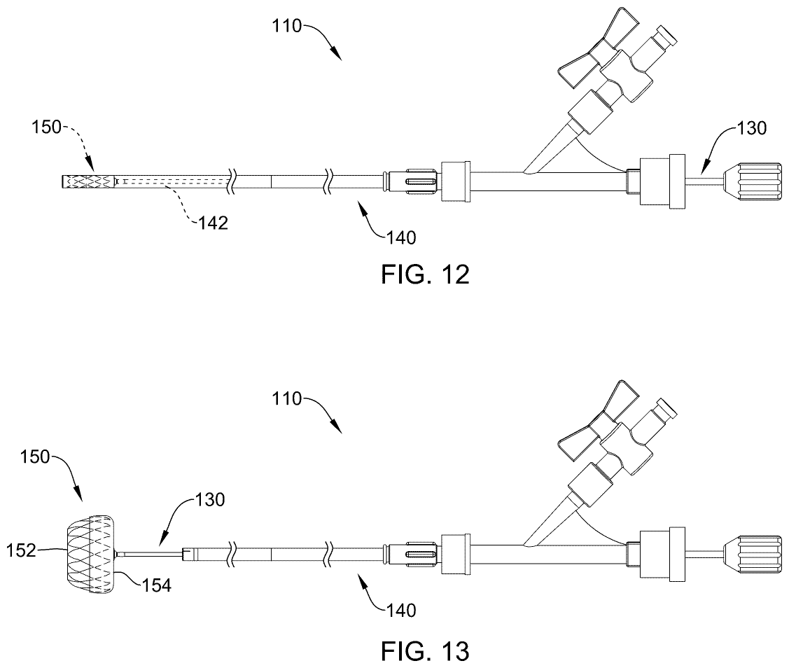

The therapeutic catheter 36 shown in FIGS. 2F through 2K may take a variety of different forms, and may be used to delivery any of a variety of different LAAC devices. FIGS. 12 and 13 schematically show selected components and/or arrangements of a medical device system 110 that may be used to deliver and/or deploy a variety of medical implants (e.g., a cardiovascular medical implant, an occlusive medical implant, etc.) to one or more locations within the anatomy of a patient including but not limited to the heart and/or the vasculature.

The medical device system 110 may include a catheter 140 having a lumen 142 extending from a proximal opening to a distal opening, a core wire 130 movably and/or slidably disposed within the lumen 142, and a medical implant 150 (e.g., a cardiovascular medical implant, an occlusive medical implant, etc.). The medical implant 150 may be configured to occlude the left atrial appendage of the patient.

The medical implant 150 may include an expandable framework 152 configured to shift between a collapsed configuration (e.g., FIG. 12), wherein the medical implant 150 is disposed within the lumen 142 proximate the distal opening in the collapsed configuration, and an expanded configuration (e.g., FIG. 13), wherein the medical implant 150 and/or the expandable framework 152 is configured to shift between the collapsed configuration and the expanded configuration when the medical implant 150 is disposed distal of the distal opening of the lumen 142 and/or the catheter 140, and/or when the medical implant 150 is unconstrained by the catheter 140. The medical implant 150 includes an occlusive element 154 that extends over at least part of the expandable framework 152.

The medical implant 150 may be disposed at and/or releasably connected to a distal portion of the core wire 130. In some embodiments, the medical implant 150 may be releasably connected to the distal end of the core wire 130. The core wire 130 may be slidably and/or rotatably disposed within the lumen 142 of the catheter 140. In some embodiments, a proximal end of the core wire 130 may extend proximally of a proximal end of the catheter 140 and/or the proximal opening of the lumen 142 for manual manipulation by a clinician or practitioner.

Some suitable, but non-limiting, examples of materials for the medical device system 110, the core wire 130, the catheter 140, and/or the medical implant 150, etc. are discussed below. It is contemplated that any and/or all medical implants disclosed herein may be used in accordance with and/or be associated with the medical device system 110 described above.

The materials that can be used for the various components of the devices described herein may include those commonly associated with medical devices. In some instances, the various components of the devices described herein may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly (alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some instances, a degree of Magnetic Resonance Imaging (MRI) compatibility is imparted into the devices and components thereof disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-NR and the like), nitinol, and the like, and others.

In some instances, the devices and components thereof may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some instances, the devices and components thereof disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni—Co—Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some instances, the devices and components thereof disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A medical device assembly, comprising:
a first elongate member adapted to be advanced through a puncture site formed within a body structure, the first elongate member including:
a first elongate shaft; and
a ferrous material within the first elongate shaft at a position intended to remain at a first side of the puncture site; and
a second elongate member adapted to be advanced through the puncture site adjacent the first elongate member, the second elongate member including:
a second elongate shaft; and
one or more magnets secured to the second elongate shaft, the one or more magnets adapted to be attracted to the ferrous material of the first elongate member in order to guide the second elongate member towards the first elongate member and through the puncture site.

2. The medical device assembly of claim 1, wherein the ferrous material within the first elongate shaft comprises an additional magnet secured to the first elongate shaft.

3. The medical device assembly of claim 1, wherein the body structure comprises an atrial septum, and the puncture site is formed in the atrial septum.

4. The medical device assembly of claim 2, wherein the additional magnet secured to the first elongate shaft comprises an elongate magnet extending along the first elongate shaft.

5. The medical device assembly of claim 2, wherein the first elongate shaft comprises a coiled elongate shaft, and the additional magnet is coiled within the coiled elongate shaft.

6. The medical device assembly of claim 2, wherein the additional magnet comprises a magnet disposed within the first elongate shaft.

7. The medical device assembly of claim 2, wherein the additional magnet comprises one or more magnets secured to the first elongate shaft.

8. The medical device assembly of claim 1, wherein the one or more magnets comprise one or more electromagnets.

9. The medical device assembly of claim 1, wherein the first elongate member comprises a guidewire.

10. The medical device assembly of claim 1, wherein the second elongate member comprises a visualization catheter.

11. The medical device assembly of claim 1, wherein the second elongate member comprises an intracardiac echocardiographic (ICE) catheter.

12. The medical device assembly of claim 1, wherein the second elongate member comprises a guidewire.

13. An assembly for accessing and visualizing a patient's left atrium, the assembly comprising:
a guidewire including:
a guidewire shaft adapted to extend through a puncture formed in the patient's atrial septum; and
a guidewire magnet secured to the guidewire shaft at a position intended to remain at a first side of the puncture; and
a visualization catheter adapted to be advanced through the puncture adjacent the guidewire, the visualization catheter including:
an elongate shaft; and
one or more visualization catheter magnets secured to the elongate shaft, the one or more visualization catheter magnets adapted to be attracted to the guidewire magnet in order to guide the visualization catheter towards the guidewire and through the puncture.

14. The assembly of claim 13, wherein the guidewire magnet comprises an elongate magnet extending along the guidewire shaft.

15. The assembly of claim 13, wherein the guidewire magnet comprises a single magnet secured to the guidewire shaft.

16. The assembly of claim 13, wherein the guidewire magnet comprises a plurality of magnets secured relative to the guidewire shaft.

17. The assembly of claim 12, wherein the visualization catheter comprises an intracardiac echocardiographic (ICE) catheter.

18. A kit for accessing and visualizing a patient's left atrium, the kit comprising:
a guidewire having a guidewire magnet secured to the guidewire at a position intended to remain at a first side of a puncture formed in the atrial septum; and
a visualization catheter adapted to be advanced through the puncture adjacent the guidewire, the visualization catheter including an elongate shaft and one or more visualization catheter magnets secured to the elongate shaft, the one or more visualization catheter magnets adapted to be attracted to the guidewire magnet in order to guide the visualization catheter towards the guidewire and through the puncture.

19. The kit of claim 18, wherein the one or more visualization catheter magnets comprise electromagnets.

20. The kit of claim 18, wherein the visualization catheter comprises an intracardiac echocardiographic (ICE) catheter.
